(19) United States
(12) Patent Application Publication
(10) Pub. No.: US 2008/0262498 A1
(43) Pub. Date: Oct. 23, 2008

(54) Double locked hip implant

(76) Inventor: Alberto Angel Fernandez Dell'Oca, Montevideo (UY)

Correspondence Address: ALBERTO FERNANDEZ DELL'OCA, DEAUVILLE 1701, MONTEVIDEO, UY

(21) Appl. No.: 11/785,436

(22) Filed: Apr. 18, 2007

(51) Int. Cl. A61B 17/58

(52) U.S. Cl. 606/65; 606/67

(57) ABSTRACT

A novel apparatus for treating fractures of the femur is disclosed. The assembly includes two hip implants positioned into the head and neck of the femur. The hip implants solidly lock into each other, while retaining the possibility of being slided together through either the oblique bore of an intramedullary nail or through the barrel of a side plate. This novel apparatus allows the surgeon to achieve sliding rotational control of the femoral head, while avoiding independent rotation of each screw around its own axis. This novel apparatus also avoids independent sliding of each screw.

### BACKGROUND OF THE INVENTION

The present invention relates to a device for use in osteosynthesis to repair femoral fractures, and in particular to a device to immobilize bone fragments of fractures occurring in the proximal region of the femur.

A variety of systems have been developed to treat proximal femoral fractures, which are basically based on a hip nail or a lag screw that is inserted from the side of the femur through the neck and into the femoral head, being afterwards fixed either to an intramedullary nail positioned inside the femoral shaft, or to a side plate positioned in the outside of the femoral shaft.

In 1960, the compression hip screw was introduced permitting improved fixation of proximal femoral fractures, allowing the surgeon to compress the bone fragments towards each other. In 1969, Zickel developed the intramedullary rod and cross nail assembly, disclosed in U.S. Pat. No. 3,433,220, consisting on an intramedullary nail located inside the marrow canal of the femoral shaft, and a cross nail that passes through the intramedullary nail and extends towards the femoral head, being fixed to the intramedullary nail by a set screw which does not allow the backing out for the cross nail. This device, while permitting an adequate fixation and rotational control of the fracture, does not allow sliding and therefore fails to provide compression of the proximal bone fragments against each other. As a result, bone contact was insufficient to support the patient's weight, resulting in an increased risk of bending or breaking of the implanted hip nail. This fact, together with the shape of the hip nail, determinate too much pressure over the femoral neck and head bone tissue, that could lead the implant to cut through the cancellous tissue of the femoral neck or head in a condition known as “cut out”, causing the nail to pierce the surface of the femoral neck or head, or at least to loose the proper alignment of the bone fracture.

To solve one of these difficulties, collapsible implants where developed, such as those disclosed in U.S. Pat. Nos. 5,176,681, 5,573,536 and 5,032,125. In these kind of implants the hip nail or screw is allowed to slide back through a bore in the side plate or intramedullary nail, permitting the migration of the bone fragments into each other, and therefore allowing the reduction of the fracture as the patient wanders, bearing weight in the fractured limb. This fact determines an increased bone contact, permitting to tolerate more pressure and therefore minimizing the tendency of breaking the implant. However, this type of implant lacks rotational control, allowing rotation of the femoral head around the hip screw.

Another femoral fracture devices, such as that disclosed in U.S. Pat. No. 5,167,663, consist in an intramedullary rod and a hip screw angled in the direction of the femoral head, with a threaded front portion that engages the femoral head and a smooth rear portion that slidably passes trough a hole in the head of the intramedullary rod to permit sliding compression of proximal femoral fracture. These devices include an optional second screw parallel to the first one, which also allows sliding compression and adds rotational control of the fracture. However, as these hip screws are not attached to each other, they have the disadvantage of permitting independent rotation around the screw axis and sliding of each screw, which may cause one screw to rotate around its own axis or slide respect to the other screw.

U.S. Pat. No. 5,151,103 discloses a plate and screws to allow blocking of conical head screws in the conical screw holes existing at the plate. However, blocking of screw head to plate hole, means zero micro-motion between both metal implants.

There is therefore a need among surgeons and other medical personnel in this field for an osteosynthetic implant to treat proximal femoral fractures that minimizes the tendency to cut through the femoral head and neck tissue after insertion, permits sliding, maintains rotational control avoiding the risk of independent rotation around screw axis or sliding of parallel hip screws, and has an easy insertion technique.

### BRIEF SUMMARY OF THE INVENTION

Is therefore an object of the present invention to provide a novel orthopedic device for minimal invasive treatment of proximal femoral fractures, which combines the advantages of intramedullary nails in fracture fixation with the benefits of sliding hip screws on fracture reduction.

Another object of the present invention is to provide a system rotationally stable that inhibits rotation of the femoral head on the axis of the hip implants.

Yet another object of the present invention is to teach an easy insertion technique of a double screw system that implies less surgical time without consuming a large area inside the femoral neck, by inserting both hip implants close together, making the insertion technique less demanding for the surgeon. The present invention by being an easy and straightforward procedure for the treatment of proximal femoral fractures, makes bone fixation of intramedullary nails simple and fast overcoming one of the most important subject of matter of actual surgery, time shortening.

A further object of the present invention is to provide a system that eliminates the postoperative complication associated with independent rotation around screw axis and independent sliding of parallel hip screws, by solidly blocking both parallel implants into one another so as to eliminate micro-motion between both parallel screws.

A still further object of the present invention is to provide a hip implant that is easy to be removed.

By fulfilling the recently mentioned objects, the present invention is extremely helpful to the medical care area.

The first embodiment of the present invention is an intramedullary double locked hip implant, which comprises an intramedullary nail and two femoral hip implants: the hip screw and the hip peg, the hip implants being rigidly affixed to one another after insertion so as to create a single mechanical unit, the double locked hip implant assembly. The intramedullary nail is preferably cannulated and is provided with an oblique opening proximate to its upper end. This oblique opening is figure eight shaped so as to accommodate both hip screws solidly affixed one over the other. The above mentioned cannulation and the oblique bore communicate in the inner part of the intramedullary nail. Both hip implants have a rear head that allows the solid attachment of one into the other by a threaded mechanism. Both hip implants have a frontal smooth shaft, which allows sliding back through the oblique opening of the intramedullary nail. Both hip implants may be of different or equal diameter, and either of them can be inserted over the other through the figure eight shaped oblique bore of the intramedullary nail, being the hip screw the first hip implant to be inserted, followed by the insertion of the hip peg. The hip screw is preferably cannulated to permit its insertion over a Kirschner wire. The head of said hip screw is provided with an internally threaded notch to engage the head of the hip peg, allowing solid fixation of both implants into one another. The shaft of the hip screw has a longitudinal groove with a dead end so as to receive and lock the shaft of the hip peg, the shaft of the hip screw having a treaded portion at its frontal end designed to be screwed into the femoral head. The rest of the shaft of the hip screw is smooth so as to allow the sliding back of the screw through the oblique opening of the intramedullary nail. The hip peg comprises an externally threaded head, which engages with the notch in the head of the hip screw, and a smooth shaft, which fits into the groove at the shaft of the hip screw, creating the double hip implant assembly, the double hip implant assembly passing through the oblique opening in the intramedullary nail. With the intramedullary nail in position within the femoral medullary channel, the hip screw is inserted to its final position in a manner consistent with common technique. Thereafter, the hip peg is inserted passing through the oblique opening in the intramedullary nail and into the groove of the hip screw. The hip peg is then screwed to the hip screw, solidly fixing both implants, constituting the double locked hip implant assembly. Said double locked hip implant assembly is solidly engaged to the femoral head by the treaded frontal end of the hip screw. Due to the smooth shaft of both implants, the double locked hip implant assembly is able to slide back through the oblique opening of the intramedullary nail, allowing the compression of bone fragments. The solid fixation mechanism of both hip implants into one another inhibits independent migration of each of the hip implants. Furthermore, the double locked hip implant assembly, by being constituted of two implants, inhibits rotation of the femoral head on the axis of said double locked hip assembly. One variation of the first embodiment has an intramedullary nail with optional conventional distal locking screws. Another variation of the first embodiment has a coaxial screw designed to prevent the hip implant assembly from sliding.

The second embodiment of the present invention is a side plated double locked hip implant. This second embodiment comprises a side plate with a barrel and two hip implants: a hip peg and a hip screw, the hip implants being solidly fixed to one another by a threaded mechanism located at the front end of each of the hip implants and a dead end located at the groove of hip screw, so that they constitute the double locked hip implant. The side plate is solidly fixed to an oblique barrel, said oblique barrel being cannulated, said cannulation being figure eight shaped so as to receive both hip implants. The oblique barrel is angled, so that when the side plate is affixed to the femoral shaft, the axis of said barrel is directed towards the axis of the femoral neck. The double locked hip implant assembly slidably passes though the figure eight shaped cannulation of the barrel of the side plate so as to allow the compression of bone fragments, while preventing independent migration of each of the hip implants and providing rotational stability.

Other objects, advantages and novel features of the present invention will become apparent from the following detailed description of the invention when considered in conjunction with the accompanying drawings.

### BRIEF DESCRIPTION OF THE SEVERAL VIEWS OF THE DRAWINGS

Preferred features of the present invention are disclosed in the accompanying drawings, wherein similar reference characters denote similar elements throughout the several views, and wherein:

FIG. 1 is an exploded perspective view of the double locked hip implant assembly according to the first embodiment of the present invention.

FIG. 2A is a perspective view of the intramedullary nail according to the present invention.

FIG. 2B is a top view of FIG. 2A.

FIG. 2C is a cross sectional view, to a larger scale, of the top portion of the intramedullary nail of FIG. 2A, taken at 2C-2C of FIG. 2B.

FIG. 3 is a perspective view of the double hip implant assembly according to the first embodiment of the present invention.

FIG. 3A is a perspective view, to a larger scale, of the threaded mechanism of FIG. 3.

FIG. 4 is a perspective view of the hip screw according to the first embodiment of the present invention.

FIG. 5 is a perspective view of the hip peg according to the first embodiment of the present invention.

FIG. 5A is a perspective view, to a larger scale, of the front portion of the hip peg of FIG. 5.

FIG. 6 is a perspective view of the hip screw passing through the intramedullary nail according to the first embodiment of the present invention.

FIG. 7 is a perspective view of the hip assembly passing through the intramedullary nail according to the first embodiment of the present invention.

FIG. 7A is partial cutaway perspective view, to a larger scale, of the hip assembly passing through the intramedullary nail with the coaxial screw on it, according to the first embodiment of the present invention.

FIG. 8 is a side view of the double locked hip implant assembly, according to the first embodiment of the present invention, at its final position.

FIG. 9 is an exploded perspective view of the double locked hip implant assembly according to the second embodiment of the present invention.

FIG. 10A is a front view of the side plate according to the second embodiment of the present invention.

FIG. 10B is a cross sectional view of the side plate taken at 10B-10B of FIG. 10A.

FIG. 10C is a perspective view of the side plate according to the second embodiment of the present invention.

FIG. 11 is a perspective view of the hip screw according to the second embodiment of the present invention.

FIG. 11A is a perspective view, to a larger scale, of the front portion of the hip screw of FIG. 11.

FIG. 11B is a side view of the hip screw of FIG. 11.

FIG. 11C is a cross sectional view of the hip screw taken at 11C-11C of FIG. 11B.

FIG. 12 is a perspective view of the hip peg according to the second embodiment of the present invention.

FIG. 12A is a perspective view, to a larger scale, of the rear portion of the hip peg of FIG. 12.

FIG. 13 is a perspective view of the double locked hip implant assembly, according to the second embodiment of the present invention, at its final position.

FIG. 13A is a perspective view, to a larger scale, of the rear portion of the double hip implant assembly of FIG. 13.

### DETAILED DESCRIPTION OF THE INVENTION

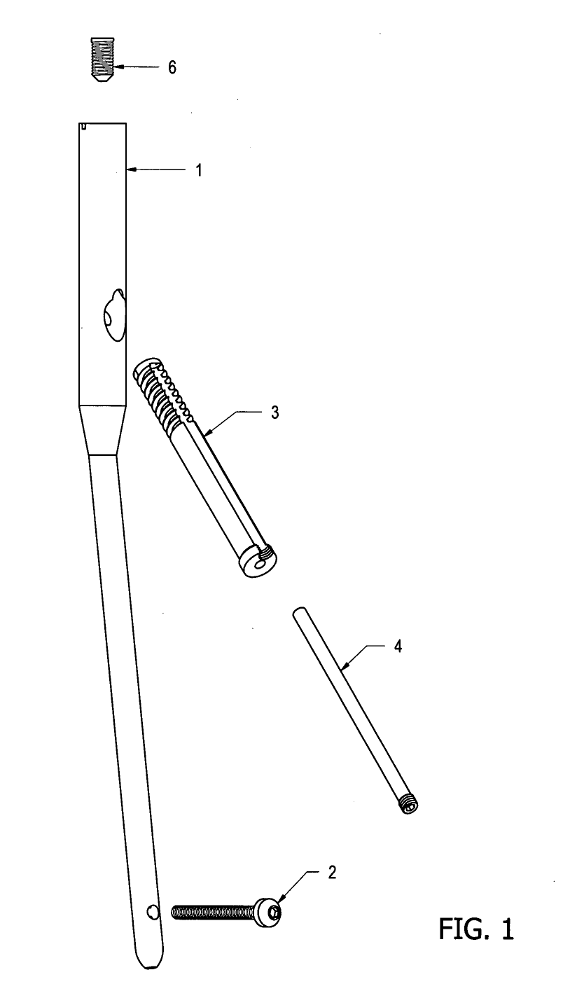

Hereinafter, a device to immobilize bone fragments of fractures occurring in the proximal region of the femur, according to the first embodiment of the present invention, will be explained with reference to FIGS. 1-8.

FIG. 1 illustrates the individual components of the first embodiment of the present invention. In the illustrated embodiment, the device includes an intramedullary nail 1 and two femoral hip implants: the hip screw 3, and the hip peg 4. The optional conventional distal locking screw 2, and an optional coaxial screw 6 are also shown.

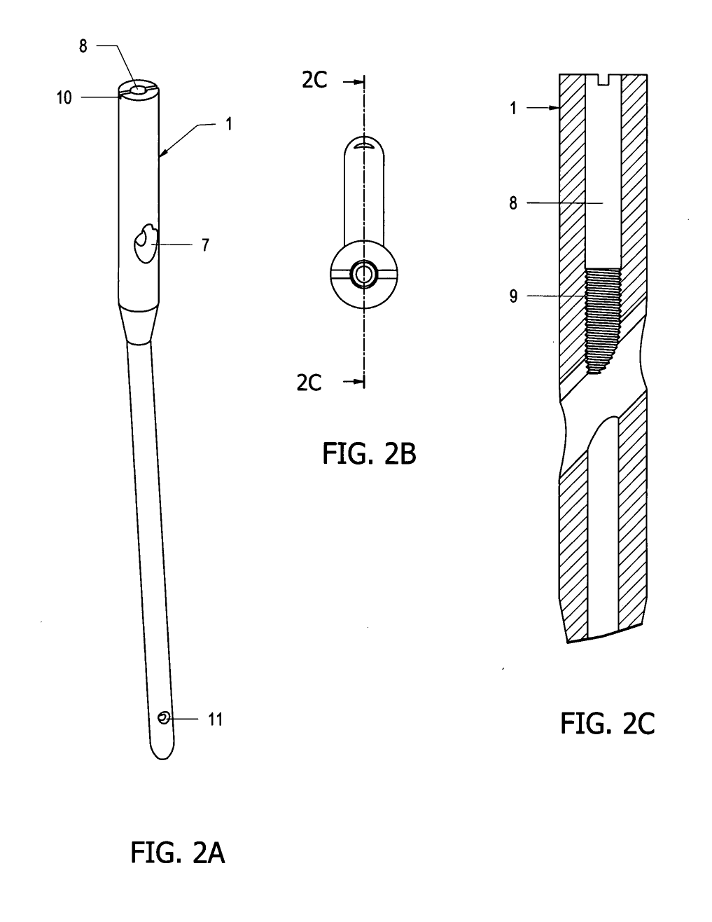

The intramedullary nail 1 is illustrated in FIGS. 2A, 2B and 2C. The intramedullary nail 1 is provided with an oblique opening 7 proximate to its upper end. This oblique opening 7 has a figure eight shape so as to receive the double hip implant assembly 5, and is angled so that when the intramedullary nail 1 is positioned inside the medullary channel, the axis of the oblique opening 7 is directed toward the axis of the femoral neck. The intramedullary nail 1 is preferably cannulated, the cannulation 8 communicating with the oblique opening 7 in the inner part of the intramedullary nail 1. The cannulation 8 is provided with an internal thread 9 designed to engage with the optional coaxial screw 6. The intramedullary nail 1 has a slot 10 at its upper end and is provided with distal transverse holes 11 to receive the optional distal locking screws 2.

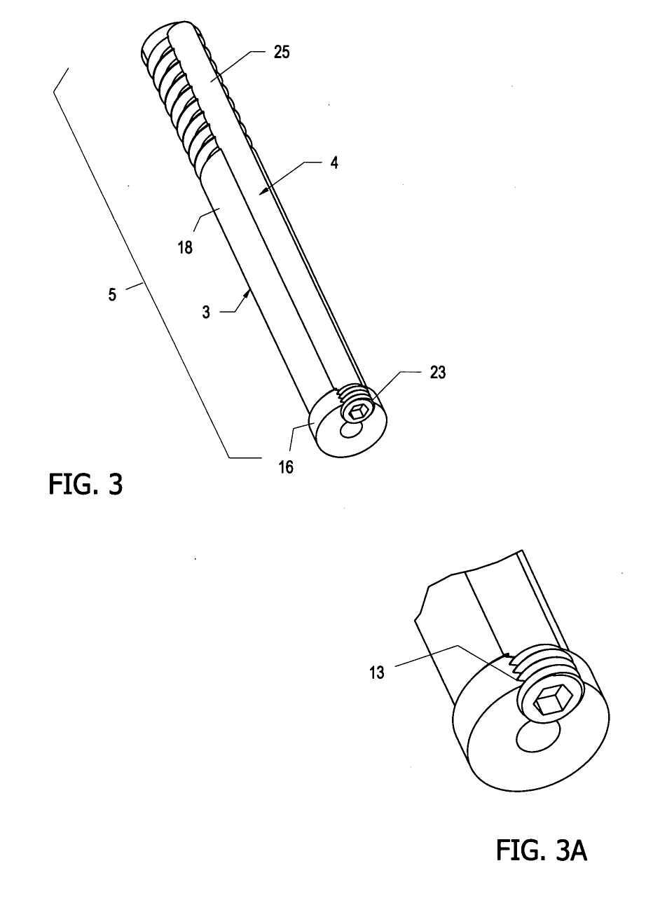

FIGS. 3 and 3A illustrate the double hip implant assembly 5. Both hip implants have a rear head 16,23, which allow the solid attachment of one implant into the other by a threaded mechanism 13, and frontal shafts 18, 25, which allow sliding back of the double hip implant assembly 5 through the oblique opening 7 of the intramedullary nail 1. Both hip implants, the hip screw 3, and the hip peg 4, may be of different or equal diameter, and either of them can be inserted over the other through the figure eight shaped oblique opening 7 of the intramedullary nail 1.

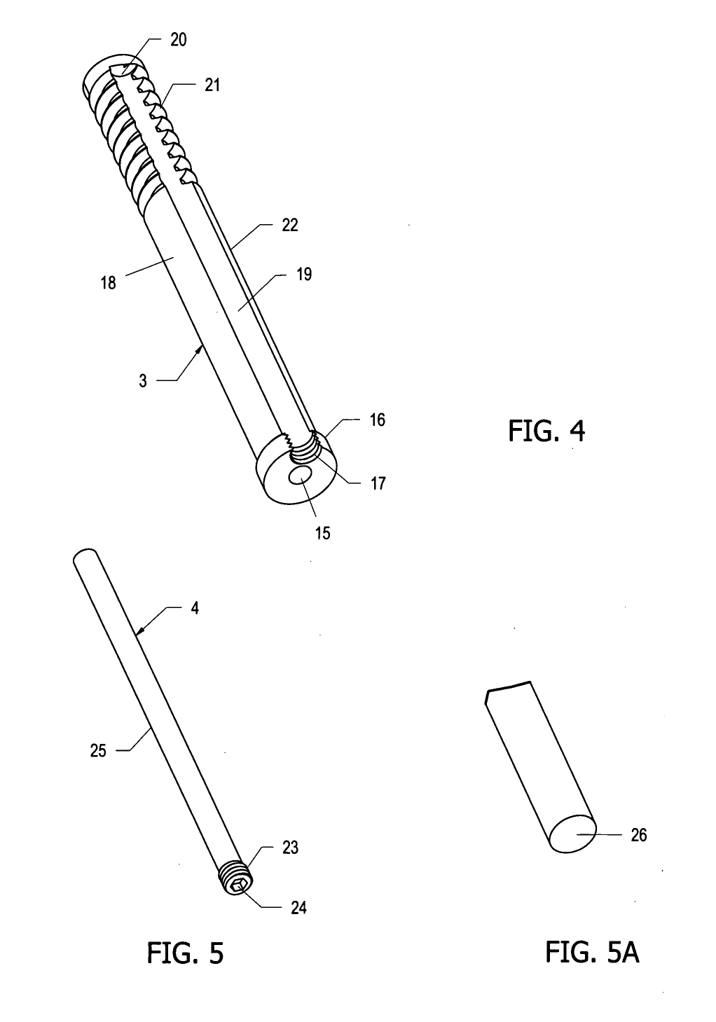

FIG. 4 depicts the hip screw 3, which is preferably cannulated 15 to permit its insertion over a Kirschner wire. The shaft 18 of the hip screw 3 has a longitudinal groove 19 to receive the shaft of the hip peg 4, said groove 19 extending from the internally threaded notch 17 towards the frontal end of the screw and terminating in a vertical surface 20, which is convex, so as to provide grater fixation of both implants into one another. The shaft 18 of the hip screw 3 has a front portion that is externally treaded 21 so as to be screwed into the femoral head, while maintaining a rear smooth portion 22 that allows the said shaft 18 to slide back through the oblique opening 7 of the intramedullary nail 1. The head 16 of said hip screw 3 is provided with an internally threaded notch 17 to engage the externally treaded head 23 of the hip peg 4, allowing solid fixation of both implants into one another, as shown in FIG. 5.

The hip peg 4 is illustrated in FIGS. 5 and 5A and consists of an externally treaded head 23, which engages with the internally treaded notch 17 of the hip screw 3, and a smooth shaft 25, which fits into the groove 19 of the hip screw 3. The shaft 25 of the hip peg 4 slidably passes through the oblique opening 7 of the intramedullary nail 1. Said shaft 25 ends in a concave surface 26 that accommodates the convex surface 20 at the end of the groove 19 of the hip screw 3. The head 23 of the hip peg 4 is provided with an hexagonal hole 24 at its rear edge designed to accommodate an hexagonal screwdriver.

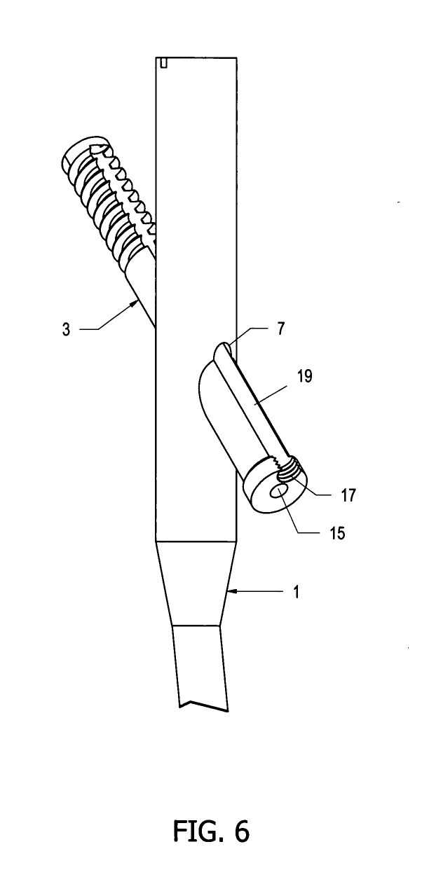

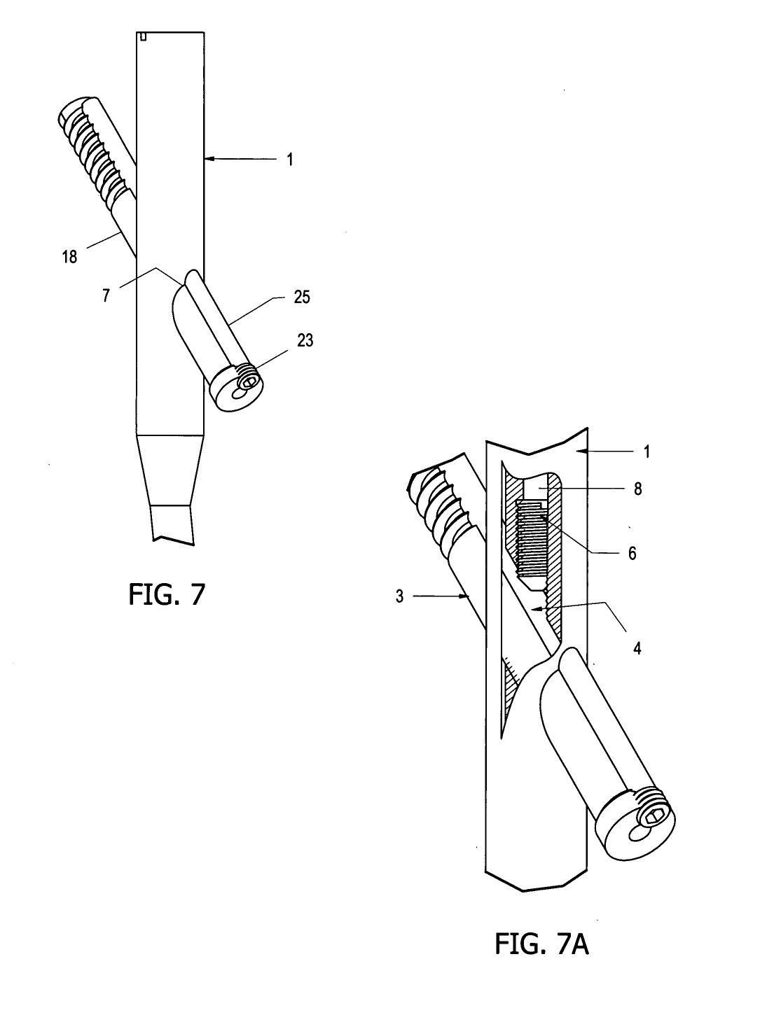

The insertion procedure is shown in FIGS. 6, 7, 7A, and 8. When the intramedullary nail 1 is positioned in the femoral medullary channel, the hip screw 3 is inserted to its final position in a manner consistent with the common technique, as illustrated in FIG. 6. Then, the hip peg 4 is inserted passing through the oblique opening 7 of the intramedullary nail 1 and through the groove 19 of the hip screw 3, as shown in FIG. 7. Then, the head 23 of the hip peg 4 is screwed to the notch 17 of the hip screw 3, solidly fixing both implants, constituting the double locked hip implant assembly 5. If needed, an optional coaxial screw 6 may be inserted into the cannulation 8 of the intramedullary nail 1, so as to tighten up the double locked hip implant assembly 5 in the inner part of the oblique opening 7 to prevent further sliding of said double hip implant assembly 5, as shown in FIG. 7A.

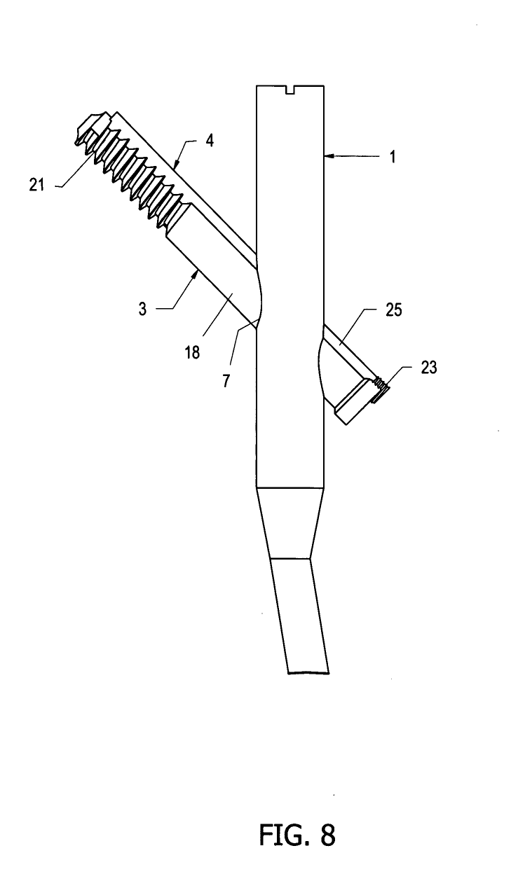

FIG. 8 depicts the double locked hip implant assembly 5 according to the first embodiment of the present invention, at its final position. The double locked hip implant assembly 5 is solidly engaged to the femoral head by the treaded frontal end 21 of the hip screw 3. Due to the smooth shaft 18 of the hip screw 3 and the smooth shaft 25 of the hip peg 4, the double locked hip implant assembly 5 is able to slide back through the oblique opening 7 of the intramedullary nail 1, allowing compression of bone fragments. The solid fixation mechanism of both hip implants 3, 4 into one another, inhibits independent migration. The double locked hip implant assembly 5, by being constituted of two hip implants 3, 4, inhibits the rotation of the femoral head around the axis of the double locked hip implant assembly 5.

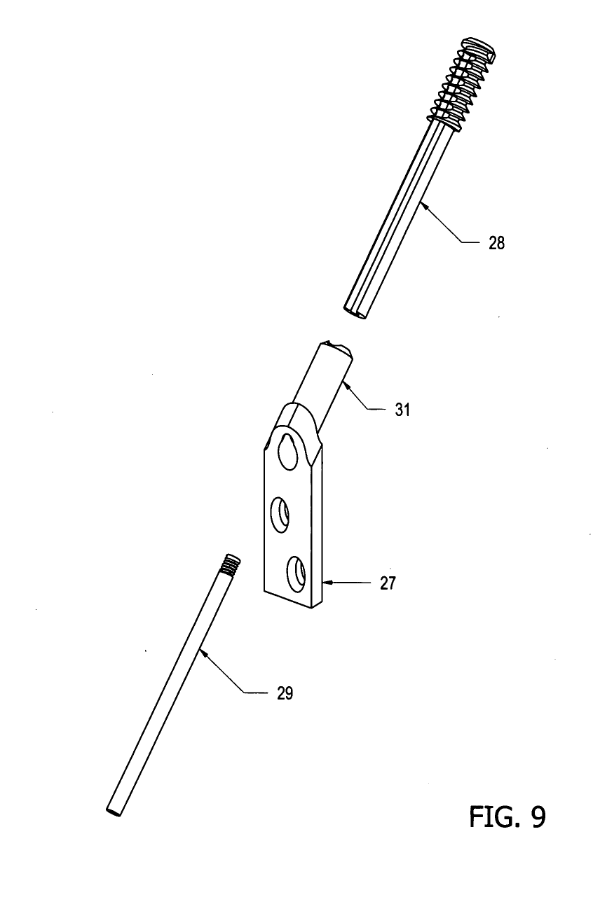

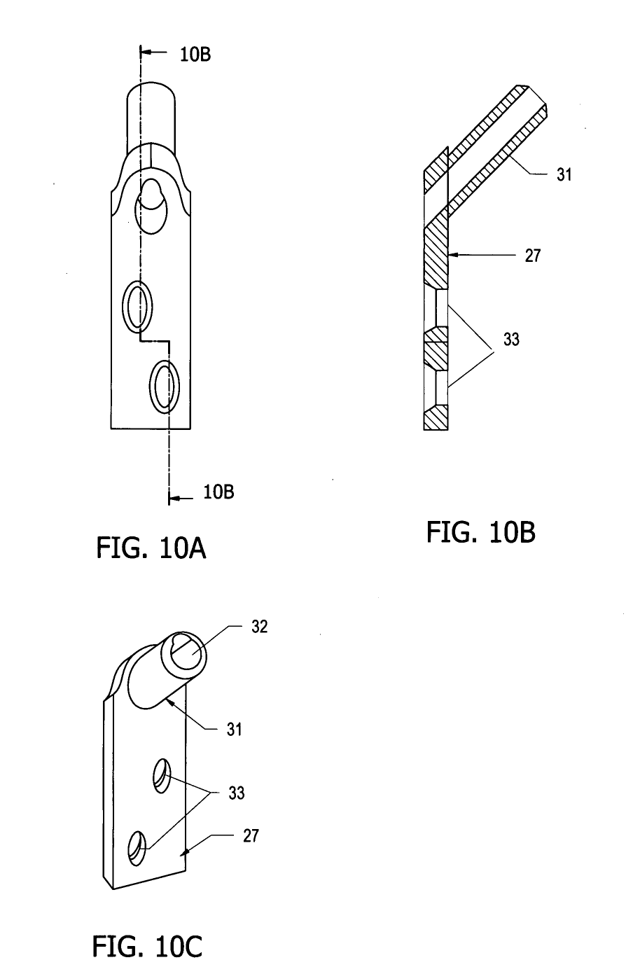

Next, a device to immobilize bone fragments of fractures occurring in the proximal region of the femur according to the second embodiment of the present invention will be explained with reference to FIGS. 9-13A.

As shown in FIG. 9, the second embodiment of the present invention consists of a side plate 27 with a barrel 31 and two femoral hip implants: the hip screw 28 and the hip peg 29.

As illustrated in 10A, 10B, and 10C, the side plate 27 consists in a plate with multiple bores 33, which receive the screws that affix the side plate 27 to the femur. At its proximal end, said side plate 27 is solidly affixed to an oblique barrel 31, which has a figure eight shaped cannulation 32. The barrel 31 is angled, so that when the side plate 27 is affixed to the femoral shaft, the axis of the barrel 31 is aligned with the axis of the femoral neck. The figure eight shaped cannulation 32 of the barrel 31 is designed to accommodate the double hip implant assembly, which slidably passes through said figure eight shaped cannulation 32.

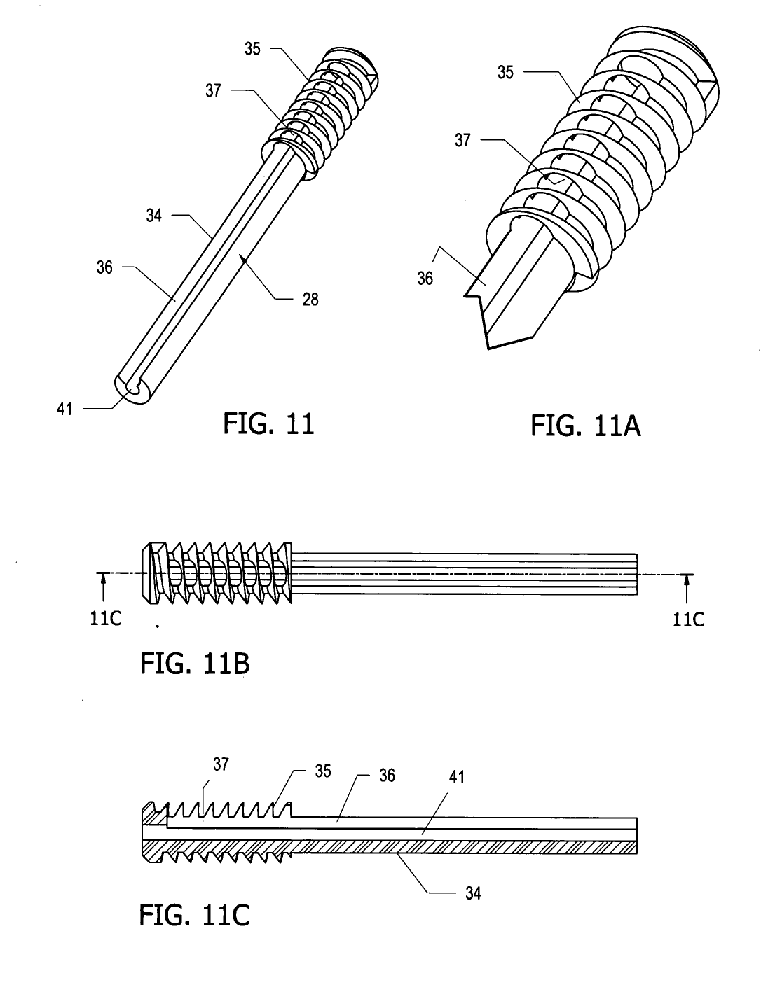

As shown in FIGS. 11, 11A, 11B, and 11C, the hip screw 28 of the second embodiment consists of a grooved shaft 34, and a front end, which is provided with an external thread 35. The shaft 34 has a longitudinal groove 36, which begins at the rear end of said shaft 34 and is continued by a threaded canal 37, extending through the external threads 35 of the front end of the hip screw 28. The longitudinal groove 36 and the threaded canal 37 are sized to accommodate the hip peg 29. The hip screw 28 is provided with a cannulation 41 designed to receive a Kirschner wire during the insertion procedure.

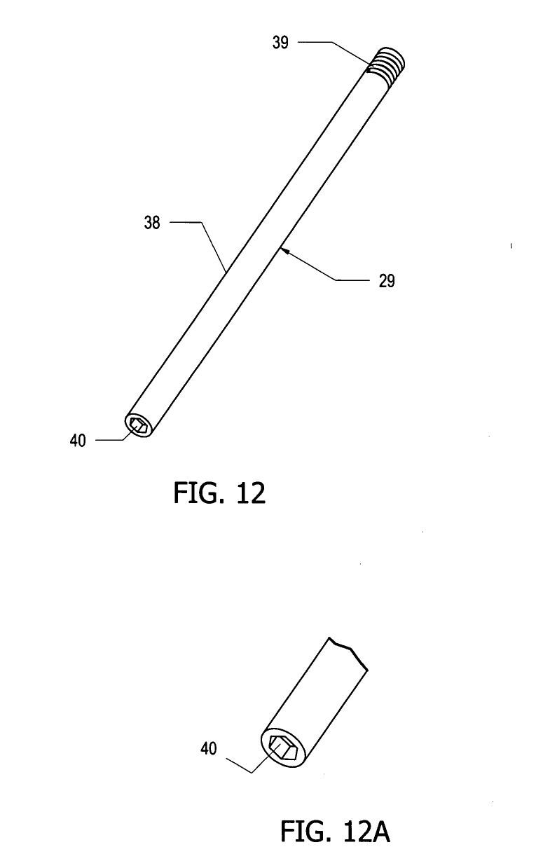

The hip peg 29 consists in a smooth shaft 38 provided with a male thread 39 at its front end, designed to engage with the female threads of the canal 37 of the hip screw 28, as shown in FIGS. 12 and 12A. The rear end of the hip peg 29 is provided with an hexagonal hole 40 designed to receive an hexagonal screw driver.

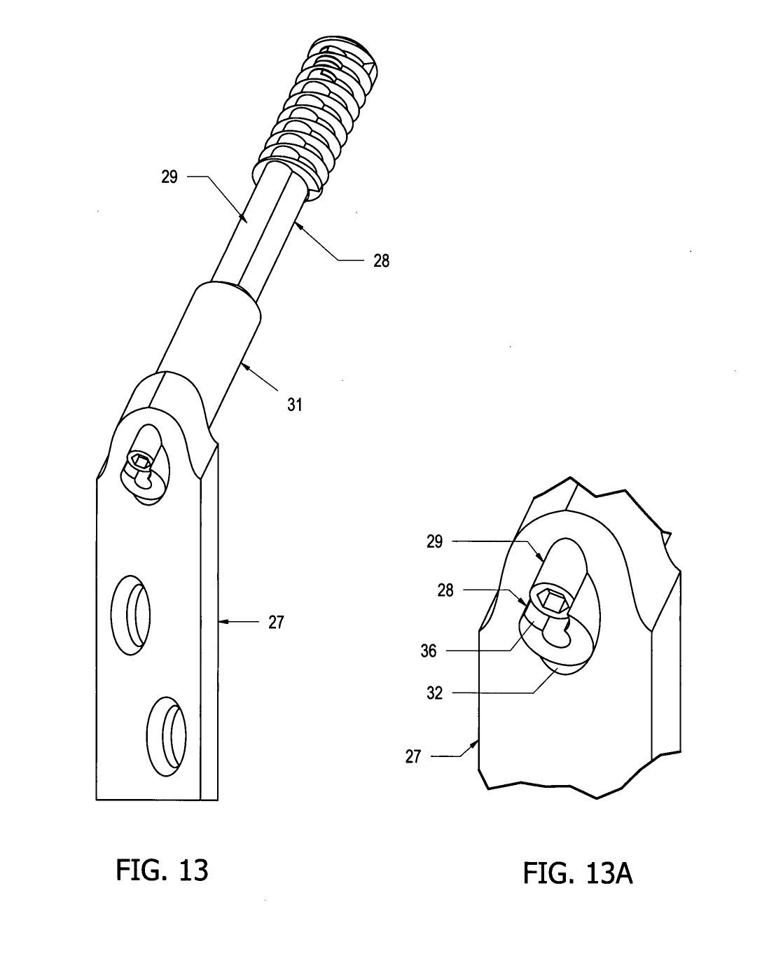

FIGS. 13 and 13A depict the double locked hip implant and the side plate 27 at their final positions. In operation, the hip screw 28 is screwed into the femoral neck in a manner consistent with the common technique. Then, the side plate 27 is inserted introducing the shaft 34 of the hip screw 28 through the cannulation 32 of the barrel 31 of the side plate 27, the side plate 27 being attached to the femoral shaft by means of bone screws. Thereafter, the hip peg 29 is inserted through the figure eight shaped cannulation 32 of the barrel 31 and into the groove 36 of the hip screw 28, the hip peg 29 being introduced until reaching the threaded canal 37 of the hip screw 28. Then, the hip peg 29 is threaded into the hip screw 28 until it reaches the dead end of the groove. This mechanism solidly affixes both implants, constituting the double locked hip implant assembly.

What I claim as my invention is:

1. An instrument assembly comprising two substantially parallel screws inserted into the head and neck of the femur, approximately parallel to the femoral neck axis, wherein:
e) one of said screws is partially nested into a groove existing on the other screw;
a) one of said screws is threaded into the other one, until reaching the dead end of the groove;
b) both said screws solidly lock into each other eliminating any micro-motion between both screws;
c) both said screws solidly lock into each other so that being impossible for each screw to rotate around its own axis with respect to the other screw;
d) both said screws solidly lock into each other so that being impossible for each screw to slide along its own axis with respect to the other screw;
f) said assembly slidably passes through a figure eight shaped oblique opening existing in an elongated implant coupled to the femoral shaft; and
g) the axis of the figure eight shaped oblique opening existing in the elongated implant is approximately parallel to the femoral neck axis.

2. The instrument assembly of claim 1, wherein said elongated implant coupled to the femoral shaft is an intramedullary nail, the intramedullary nail having a thicker proximal part with a figure eight shaped oblique opening sized to slidably receive the said assembly.

3. The instrument assembly of claim 1, wherein said elongated implant coupled to the femoral shaft is a side plate provided of a barrel, the barrel being sized to slidably receive said assembly.
